SEQUENCE LISTING (15 sequences)

SEQ ID NO: 1 - DNA, 693 bp, Artificial Sequence
  Synthesized
atggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactctcggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttga

SEQ ID NO: 2 - protein, 230 aa, Artificial Sequence
  Synthesized
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSRHWFMLMPKQKVAGPLCIRMDQAIMDKNIILKANFSVIFDRLETLILLRAFTEEGTIVGEISPLPSLPGHTDEDVKNAVGVLIGGLEWNNNTVRVSETLQRFAWRSSNENGRPPLTPKQKRKMAGTIRSEV

SEQ ID NO: 3 - DNA, 2202 bp, Artificial Sequence
  Synthesized
ctgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattgattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaa

SEQ ID NO: 4 - DNA, 2303 bp, Artificial Sequence
  Synthesized
atttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaacttagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatga

SEQ ID NO: 5 - DNA, 2290 bp, Artificial Sequence
  Synthesized
atggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggagtaaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggaggtcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtggaatgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatag

SEQ ID NO: 6 - DNA, 1725 bp, Artificial Sequence
  Synthesized
ccaaaatgaaggcaaaactactggtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtaaattaaaaggaatagccccactacaattggggaaatgtaacatcatcggatggctcttgggaaatccagaatgcgactcactgcttccagcgagatcatggtcctacattgtagaaacaccaaactctgagaatggagcatgttatccaggagatttcatcgactatgaggaactgagggagcaattgagctcagtatcatcattagaaagattcgaaatatttcccaaggaaagttcatggcccaaccacacattcaacggagtaacagcatcatgctcccataggggaaaaagcagtttttacagaaatttgctatggctgacgaagaagggggattcatacccaaagctgaccaattcctatgtgaacaataaagggaaagaagtccttgtactatggggtgttcatcacccgtctagcagtgatgagcaacagagtctctatagtaatggaaatgcttatgtctctgtagcgtcttcaaattataacaggagattcaccccggaaatagctgcaaggcccaaagtaaaagatcaacatgggaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacgggattacaaacaaggtgaactctattatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatggaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagattaggatttcagaaatataa

SEQ ID NO: 7 - DNA, 1526 bp, Artificial Sequence
  Synthesized
tcactcacagagtgacatcgaaatcatggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaaagaa

SEQ ID NO: 8 - DNA, 1368 bp, Artificial Sequence
  Synthesized
atgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaacggcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcaatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggtaatggtgtttggataggaaggactaaaagtgacagttccagacatgggtttgagatgatttgggatcctaatggatggacagagactgatagtaggttctctatgagacaagatgttgtggcaatgactgatcggtcagggtacagcggaagtttcgttcaacatcctgagctaacagggctagactgtatgaggccttgcttctgggttgaattaatcagggggctacctgaggagaacgcaatctggactagtgggagcatcatttctttttgtggtgtgaatagtgatactgtagattggtcttggccagacggtgctgagttgccgttcaccattgacaagtagtttgtt

SEQ ID NO: 9 - DNA, 984 bp, Artificial Sequence
  Synthesized
atgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccgttgggactcatcctagctccagtgctggtctaaaagatgatcttcttgaaaatttgcaggcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaaaa

SEQ ID NO: 10 - DNA, 25 bp, Artificial Sequence
  Synthesized
gacttctgatctgcgcgaagccgat

SEQ ID NO: 11 - DNA, 25 bp, Artificial Sequence
  Synthesized
attccttgatcggcttcgcgcagat

SEQ ID NO: 12 - DNA, 25 bp, Artificial Sequence
  Synthesized
tcctcttagggatgcctgatctcgg

SEQ ID NO: 13 - DNA, 25 bp, Artificial Sequence
  Synthesized
ttcgccgagatcaggcatccctaag

SEQ ID NO: 14 - DNA, 25 bp, Artificial Sequence
  Synthesized
ctcttagggatgcctgatctgcgcg

SEQ ID NO: 15 - DNA, 25 bp, Artificial Sequence
  Synthesized
ttcgcgcagatcaggcatccctaag

(19) United States
(12) Patent Application Publication
(10) Pub. No.: US 2020/0385431 A1
(43) Pub. Date: Dec. 10, 2020

(54) REPLICATION-LIMITED MUCOSAL IMMUNE VACCINE FOR INFLUENZA VIRUS

(71) Applicant: Institute of Microbiology, Chinese Academy of Sciences, Beijing (CN)

(72) Inventors: Wenjun LIU, Beijing (CN); Jing LI, Beijing (CN); Can CHEN, Beijing (CN); Shuang ZHANG, Beijing (CN); Lei SUN, Beijing (CN); Ruifeng ZHANG, Beijing (CN); Wenhui FAN, Beijing (CN); Limin YANG, Beijing (CN)

(21) Appl. No.: 16/769,974

(22) PCT Filed: Dec. 11, 2017
(86) PCT No.: PCT/CN2017/115406

§ 371 Date: Jun. 4, 2020

(30) Foreign Application Priority Data
Dec. 6, 2017 (CN) 201711276348.3

(51) Int. Cl. C07K 14/005; C12N 7/00; A61P 31/16

(52) U.S. Cl. CPC C07K 14/005; C12N 7/00; A61P 31/16; A61K 39/00; C12N 2760/16121; C12N 2760/16162; C12N 2760/16122

(57) ABSTRACT

The present invention discloses a replication-limited mucosal immune vaccine for influenza virus. The present invention first protects a mutant protein, which is obtained by mutating two amino acid residues of NS1 protein of influenza virus as follows: the amino acid residue at position 38 is mutated from arginine to alanine, and the amino acid residue at position 41 is mutated from lysine to alanine. The present invention also protects a recombinant virus, which is obtained by mutating the codons encoding two amino acid residues of NS1 protein in the influenza virus genome as follows: the codon for the amino acid residue at position 38 from the N-terminus is mutated from the arginine codon to an alanine codon, and the codon for the amino acid residue at position 41 from the N-terminus is mutated from the lysine codon to an alanine codon. The present invention also protects use of any one of the above-mentioned recombinant viruses for preparing a vaccine for influenza virus. The present invention has great application value for the prevention and treatment of influenza virus.

### TECHNICAL FIELD

The present invention relates to a replication-limited mucosal immune vaccine for influenza virus.

### BACKGROUND ART

Influenza virus is a representative species of the genus Influenza virus of family Orthomyxoviridae, including human influenza virus and animal influenza virus. Human influenza viruses are divided into three types: A, B and C, which are the pathogens of influenza (flu). Influenza A virus is prone to mutations in antigenicity and has caused a world pandemic many times, such as the 1918-1919 pandemic, in which at least 20 million to 40 million people died of influenza worldwide. Influenza B virus is less pathogenic to humans. Influenza C virus causes only insignificant or mild upper respiratory tract infections in humans and rarely causes epidemics. Influenza A virus was successfully isolated in 1933, influenza B virus was obtained in 1940, and influenza C virus was not successfully isolated until 1949.

The RNA of influenza A virus is composed of eight segments, wherein the first, second and third segments encode the RNA polymerase, the fourth segment is responsible for encoding the hemagglutinin, the fifth segment is responsible for encoding the nucleoprotein, the sixth segment encodes the neuraminidase, the seventh segment encodes the matrix proteins, and the eighth segment encodes a non-structural protein with unknown function that can function as a splicing RNA.

### SUMMARY OF THE INVENTION

The object of the present invention is to provide a replication-limited mucosal immune vaccine for influenza virus.

The present invention first protects a mutant protein, named NSR38A/K41A protein, which is obtained by mutating two amino acid residues of NS1 protein of influenza virus as follows: the amino acid residue at position 38 is mutated from arginine to alanine, and the amino acid residue at position 41 is mutated from lysine to alanine.

The gene encoding the mutant protein (named NS1R38A/K41A gene) also belongs to the protection scope of the present invention.

The NS1R38A/K41A gene can be specifically obtained by performing the following two sets of mutations on the gene encoding the NS1 protein: the nucleotides at positions 112-114 are mutated from “cga” to “GCA”, and the nucleotides at positions 121-123 are mutated from “aag” to “GC A”.

The gene encoding the NS1 protein is specifically as shown in SEQ ID NO: 1 in the Sequence Listing.

Plasmids containing the NSIR38A/K41A gene also belong to the protection scope of the present invention. The plasmid can specifically be plasmid pHH21-NS1R38A/K41A. The plasmid pHH21-NSR38A/K41A is a recombinant plasmid obtained by inserting the NSlR38A/K41A gene into the multiple cloning site (for example, BsmBI restriction enzyme cutting site) of the vector pHH21.

The present invention further protects a recombinant virus, which is obtained by mutating the codons encoding two amino acid residues of NS1 protein in the influenza virus genome as follows: the codon for the amino acid residue at position 38 from the N-terminus is mutated from the arginine codon to an alanine codon, and the codon for the amino acid residue at position 41 from the N-terminus is mutated from the lysine codon to an alanine codon.

The recombinant virus can specifically be a recombinant virus obtained by mutating the codons encoding two amino acid residues of NS1 protein in the influenza virus genome as follows: the codon for the amino acid residue at position 38 from the N-terminus is mutated from the arginine codon “cga” to the alanine codon “GCA”, and the codon for the amino acid residue at position 41 from the N-terminus is mutated from the lysine codon “aag” to the alanine codon “GCA”.

The recombinant virus can specifically be obtained by performing the following two sets of mutations on the gene encoding the NS1 protein as shown in SEQ ID NO: 1 in the Sequence Listing in the influenza virus genome: the nucleotides at positions 112-114 are mutated from “cga” to “GCA”, and the nucleotides at positions 121-123 are mutated from “aag” to “GCA”.

The NS1 protein described above is the following (a) or (b):

(a) a protein consisting of the amino acid sequence as shown in SEQ ID NO: 2 in the Sequence Listing;

(b) a protein derived from (al) through substitution and/or deletion and/or addition of one or several amino acid residues and having the same function.

The present invention further protects a recombinant virus, and its preparation method comprises the following steps:

ex vivo mammalian cells are co-transfected with plasmid pHH21-PA, plasmid pHH21-PB1, plasmid pHH21-PB2, plasmid pHH21-HA, plasmid pHH21-NP, plasmid pHH21-NA, plasmid pHH21-M, plasmid pHH21-NS1R38A/K41A, plasmid pcDNA3.0-PA, plasmid pcDNA3.0-PB1, plasmid pcDNA3.0-PB2 and plasmid pcDNA3.0-NP and then cultured to obtain the recombinant virus.

The plasmid pHH21-PA is a recombinant plasmid obtained by inserting the double-stranded DNA molecule as shown in SEQ ID NO: 3 in the Sequence Listing into the multiple cloning site (for example, BsmBI restriction enzyme cutting site) of vector pHH21: the plasmid pHH21-PB1 is a recombinant plasmid obtained by inserting the double-stranded DNA molecule as shown in SEQ ID NO: 4 in the Sequence Listing into the multiple cloning site (for example, BsmBI restriction enzyme cutting site) of vector pHH21; the plasmid pHH21-PB2 is a recombinant plasmid obtained by inserting the double-stranded DNA molecule as shown in SEQ ID NO: 5 in the Sequence Listing into the multiple cloning site (for example, BsmBI restriction enzyme cutting site) of vector pHH21; the plasmid pHH21-HA is a recombinant plasmid obtained by inserting the double-stranded DNA molecule as shown in SEQ ID NO: 6 in the Sequence Listing into the multiple cloning site (for example, BsmBI restriction enzyme cutting site) of vector pHH21; the plasmid pHH21-NP is a recombinant plasmid obtained by inserting the double-stranded DNA molecule as shown in SEQ ID NO: 7 in the Sequence Listing into the multiple cloning site (for example, BsmBI restriction enzyme cutting site) of vector pHH21; the plasmid pHH21-NA is a recombinant plasmid obtained by inserting the double-stranded DNA molecule as shown in SEQ ID NO: 8 in the Sequence Listing into the multiple cloning site (for example, BsmBI restriction enzyme cutting site) of vector pHH21; the plasmid pHH21-M is a recombinant plasmid obtained by inserting the double-stranded DNA molecule as shown in SEQ ID NO: 9 in the Sequence Listing into the multiple cloning site (for example, BsmBI restriction enzyme cutting site) of vector pHH21; the plasmid pHH21-NS1R38A/K41A is a recombinant plasmid obtained by inserting the NS1R38A/K41A gene into the multiple cloning site (for example, BsmBI restriction enzyme cutting site) of vector pHH21; the plasmid pcDNA3.0-PA is a recombinant plasmid obtained by inserting the double-stranded DNA molecule as shown in SEQ ID NO: 3 in the Sequence Listing into the multiple cloning site (for example, between the KpnI restriction enzyme cutting site and the XhoI restriction enzyme cutting site) of vector pcDNA3.0; the plasmid pcDNA3.0-PB1 is a recombinant plasmid obtained by inserting the double-stranded DNA molecule as shown in SEQ ID NO: 4 in the Sequence Listing into the multiple cloning site (for example, between the KpnI restriction enzyme cutting site and the XhoI restriction enzyme cutting site) of vector pcDNA3.0; the plasmid pcDNA3.0-PB2 is a recombinant plasmid obtained by inserting the double-stranded DNA molecule as shown in SEQ ID NO: 5 in the Sequence Listing into the multiple cloning site (for example, between the KpnI restriction enzyme cutting site and the XhoI restriction enzyme cutting site) of vector pcDNA3.0; the plasmid pcDNA3.0-NP is a recombinant plasmid obtained by inserting the double-stranded DNA molecule as shown in SEQ ID NO: 7 in the Sequence Listing into the multiple cloning site (for example, between the KpnI restriction enzyme cutting site and the XhoI restriction enzyme cutting site) of vector pcDNA3.0.

The mammalian cell can specifically be a 293T RIG-I KO cell.

The culture conditions can specifically be 6 to 72 h at 37° C.

The present invention further protects a method for preparing a vaccine for influenza virus, comprising the following steps:

the recombinant virus is passaged N times; N is a natural number greater than or equal to 3;

the method of the first passage is as follows: mammalian cells are infected with the recombinant virus, and then cultured to collect the supernatant, which is a virus solution containing F1 generation virus:

the method of the second passage is as follows: mammalian cells are infected with the virus solution obtained from the previous passage, and then cultured to collect the supernatant, which is a virus solution containing F2 generation virus; a series of passages is performed by repeating the method of the second passage; in the N−1th passage, the collected supernatant has virus replication ability; in the Nth passage, the collected supernatant does not have virus replication ability; the supernatant collected from the N−1th passage is used as the active ingredient of the vaccine for influenza virus.

The present invention further protects a method for preparing a vaccine for influenza virus (method A), comprising the following steps:

(1) MDCK cells are infected with the recombinant virus, and then cultured to collect the supernatant, which is a F1 generation virus solution;

(2) MDCK cells are infected with the F1 generation virus solution, and then cultured to collect the supernatant, which is a F2 generation virus solution;

(3) MDCK cells are infected with the F2 generation virus solution, and then cultured to collect the supernatant, which is a F3 generation virus solution;

(4) the F3 generation virus solution is used as the active ingredient of the vaccine for influenza virus.

The present invention further protects a method for preparing a vaccine for influenza virus (method B), comprising the following steps:

(1) MDCK cells are infected with the recombinant virus, and then cultured to collect the supernatant, which is a F1 generation virus solution;

(2) A549 cells are infected with the F1 generation virus solution, and then cultured to collect the supernatant, which is a F2 generation virus solution;

(3) the F2 generation virus solution is used as the active ingredient of the vaccine for influenza virus.

The vaccine for influenza virus prepared by any one of the above methods also belongs to the protection scope of the present invention.

The present invention further protects a kit for preparing a vaccine for influenza virus, including any one of the above-mentioned recombinant viruses.

The kit also includes 293T RIG-I KO cells and/or MDCK cells and/or A549 cells and/or Vero cells.

The present invention further protects use of any one of the above-mentioned recombinant viruses for preparing a vaccine for influenza virus.

The present invention further protects use of the plasmid pHH21-PA, the plasmid pHH21-PB1, the plasmid pHH21-PB2, the plasmid pHH21-HA, the plasmid pHH21-NP, the plasmid pHH21-NA, the plasmid pHH21-M, the plasmid pHH21-NSR38A/K41A, the plasmid pcDNA3.0-PA, the plasmid pcDNA3.0-PB1, the plasmid pcDNA3.0-PB2 and the plasmid pcDNA3.0-NP for preparing a vaccine for influenza virus.

The present invention further protects use of the vaccine for preventing and/or treating influenza.

The influenza is a cold caused by an influenza virus.

Any one of the above-mentioned influenza viruses can specifically be an influenza A virus.

Any one of the above-mentioned influenza viruses can specifically be an influenza virus that infects animals.

Any one of the above-mentioned influenza viruses can specifically be an influenza virus that infects poultry or livestock.

The influenza A virus can specifically be the WSN virus A/WSN/1933 (H1N1) strain.

### DESCRIPTION OF THE DRAWINGS

FIG. 1 shows the western blot results in Example 1.

FIG. 2 shows the western blot results of F2 cells in Example 2.

FIG. 3 shows the western blot results of F3 cells in Example 2.

FIG. 4 shows the western blot results of F4 cells in Example 2.

FIG. 5 shows the western blot results of F2 cells in Example 3.

FIG. 6 shows the western blot results of F3 cells in Example 3.

FIG. 7 shows the western blot results of F4 cells in Example 3.

### DETAILED DESCRIPTION OF THE INVENTION

The following examples facilitate a better understanding of the present invention, but do not limit the present invention. Unless otherwise specified, the experimental methods in the following examples are conventional methods. Unless otherwise specified, the test materials used in the following examples were purchased from conventional biochemical reagent stores. In the following examples, each quantitative experiment was repeated three times, and the results were averaged. The PBS buffer used in the examples is PBS buffer (pH 7.2-7.4, 0.01M) unless otherwise specified.

Vector pHH21: Neumann, G. et al., Generation of influenza A viruses entirely from cloned cDNAs. P Natl Acad Sci Usa 96 (16), 9345 (1999).

Vector pcDNA3.0: Shanghai CPG Biotech Co., Ltd., catalog number: CPC030. Lipofectamine 2000: Invitrogen. 293T RIG-I KO cells: Procell Life Science &Technology Co., Ltd., catalog number: CL-0001. MDCK cells: ATCC, CCL-34. A549 cells: ATCC, CCL-185. Vero cells: Beijing Zhongke Quality Inspection Biotechnology Co., Ltd., catalog number: V0180000.

Virus infection solution: serum-free DMEM medium containing 2 μg/ml TPCK-treated pancreatin (pancreatin was added in the form of pancreatin mother liquor, pancreatin mother liquor was a solution of pancreatin with a concentration of 0.25 g/100 mL prepared in PBS buffer), 100 U/ml penicillin and 100 U/m streptomycin.

The method of plaque identification: (1) MDCK cells were seeded in a 12-well plate (approximately 1×10⁵ cells per well), cultured in a 37° C., 5% CO₂ incubator for 12 h, and the supernatant was aspirated and discarded and the cells were washed with PBS buffer; (2) the supernatant to be tested was subject to gradient dilution with the virus infection solution and respectively added to each well obtained in step (1) (three replicate wells for each dilution), incubated at 37° C. for 1 h, the supernatant was aspirated and discarded and the cells were washed with PBS buffer; (3) after completing step (2), 1 ml of mixed solution was add to each well (the preparation method of the mixed solution was as follows: one volume of 3 g/100 ml low-melting agarose aqueous solution, which was cooled to about 37° C. after melting, was mixed with one volume of phenol red-free DMEM medium, which was preheated to 37° C., and TPCK-treated pancreatin, penicillin and streptomycin were added to the mixture, so that the concentration of pancreatin was 2 μg/ml, the concentrations of penicillin and streptomycin were both 100 U/ml): (4) after completing step (3), the 12-well plate was placed at 4° C. for more than 15 min, and after the agar solidified, the well plate was turned upside down and incubated in a 37° C. incubator, the cytopathy was observed under a microscope and after 3 days of incubation, the 12-well plate was removed from the incubator and the number of plaque was counted.

The detection method of virus-serum neutralizing antibody valence: (1) MDCK cells were seeded in a 96-well plate (approximately 4×10⁴ cells per well), cultured for 12 h, and the supernatant was aspirated and discarded and the cells were washed with PBS buffer; (2) one volume of serum was mixed with four volumes of RDE enzyme (receptor destroying enzyme), incubated at 37° C. for 16 h, then inactivated in a water bath at 56° C. for 30 min, then one volume of 20% chicken red blood cells was added and mixed, incubated at 4° C. for 12 h, then centrifuged at 1000 g and the supernatant was collected, which was the pretreated serum; (3) the pretreated serum obtained in step (2) was subject to gradient dilution with DMEM medium to obtain the diluents of each antibody; (4) the WT-F1 generation supernatant prepared in Example 1 was diluted with DMEM medium to obtain a virus diluent; (5) the antibody diluent was mixed with the virus diluent (virus content: 100TCID50) in equal volumes, incubated at 37° C. for 1 h, then added to the 96-well plate obtained in step (1) with 100 μL/well, and subjected to static incubation for 1 h; (6) after completing step (5), the supernatant was aspirated and discarded and the cells were washed with PBS buffer, DMEM medium containing 2 μg/p TPCK-trypsin was added, and subjected to static culture for 72 h; (7) the number of wells of positive infected cells was observed, and the neutralizing valence of serum was calculated by Reed-Muench method.

NS1-R38A-F:
5′-GACTTCTGATCTGCGCGAAGCCGAT-3′;
NS1-R38A-R:
5′-ATTCCTTGATCGGCTTCGCGCAGAT-3′.
NS1-K41A-F:
5′-TCCTCTTAGGGATGCCTGATCTCGG-3′;
NS1-K41A-R:
5′-TTCGCCGAGATCAGGCATCCCTAAG-3′.
NS1-R38A/K41A-F:
5′-CTCTTAGGGATGCCTGATCTGCGCG-3′;
NS1-R38A/K41A-R:
5′-TTCGCGCAGATCAGGCATCCCTAAG-3′.

### Example 1. Preparation of Mutant Viruses

1. Construction of Recombinant Plasmids

The NS1 protein is as shown in SEQ ID NO: 2 in the Sequence Listing. The gene encoding the NS1 protein was named NS1 gene. The open reading frame in the NS1 gene cDNA is as shown in SEQ ID NO: 1 in the Sequence Listing. The double-stranded DNA molecule as shown in SEQ ID NO: 1 in the Sequence Listing was inserted into the BsmBI restriction enzyme cutting site of the vector pHH21 to obtain plasmid pHH21-NS1.

Using the plasmid pHH21-NS1 as a template, a single point mutation was introduced with a primer pair consisting of NS1-R38A-F and NS1-R38A-R to obtain plasmid pHH21-NS1R38A. After sequencing, compared with the plasmid pHH21-NS1, the plasmid pHH21-NSIR38A differed only in that the codon encoding the amino acid residue at position 38 from the N-terminus of the NS1 protein was mutated from the arginine codon “cga” to the alanine codon “GCA”.

Using the plasmid pHH21-NS1 as a template, a single point mutation was introduced with a primer pair consisting of NS1-K41A-F and NS1-K41A-R to obtain plasmid pHH21-NS1K41A. After sequencing, compared with the plasmid pHH21-NS1, the plasmid pHH21-NS1K41A differed only in that the codon encoding the amino acid residue at position 41 from the N-terminus of the NS1 protein was mutated from the lysine codon “aag” to the alanine codon “GCA”.

Using the plasmid pHH21-NS1 as a template, double point mutations were introduced with NS1-R38A/K41A-F and NS1-R38A/K41A-R to obtain plasmid pHH21-NSIR38A/K41A. After sequencing, compared with the plasmid pHH21-NS1, the plasmid pHH21-NSR38A/K41A differed only in that the codons encoding two amino acid residues of the NS1 protein were mutated as follows: the codon encoding the amino acid residue at position 38 from the N-terminus was mutated from the arginine codon “cga” to the alanine codon “GCA” and the codon encoding the amino acid residue at position 41 from the N-terminus was mutated from the lysine codon “aag” to the alanine codon “GCA”.

The double-stranded DNA molecule as shown in SEQ ID NO: 3 in the Sequence Listing was inserted into the BsmBI restriction enzyme cutting site of the vector pHH21 to obtain plasmid pHH21-PA.

The double-stranded DNA molecule as shown in SEQ ID NO: 4 in the Sequence Listing was inserted into the BsmBI restriction enzyme cutting site of the vector pHH21 to obtain plasmid pHH21-PB1.

The double-stranded DNA molecule as shown in SEQ ID NO: 5 in the Sequence Listing was inserted into the BsmBI restriction enzyme cutting site of the vector pHH21 to obtain plasmid pHH21-PB2.

The double-stranded DNA molecule as shown in SEQ ID NO: 6 in the Sequence Listing was inserted into the BsmBI restriction enzyme cutting site of the vector pHH21 to obtain plasmid pHH21-HA.

The double-stranded DNA molecule as shown in SEQ ID NO: 7 in the Sequence Listing was inserted into the BsmBI restriction enzyme cutting site of the vector pHH21 to obtain plasmid pHH21-NP.

The double-stranded DNA molecule as shown in SEQ ID NO: 8 in the Sequence Listing was inserted into the BsmBI restriction enzyme cutting site of the vector pHH21 to obtain plasmid pHH21-NA.

The double-stranded DNA molecule as shown in SEQ ID NO: 9 in the Sequence Listing was inserted into the BsmBI restriction enzyme cutting site of the vector pHH21 to obtain plasmid pHH21-M.

The double-stranded DNA molecule as shown in SEQ ID NO: 3 in the Sequence Listing was inserted between the KpnI restriction enzyme cutting site and the XhoI restriction enzyme cutting site of the vector pcDNA3.0 to obtain plasmid pcDNA3.0-PA.

The double-stranded DNA molecule as shown in SEQ ID NO: 4 in the Sequence Listing was inserted between the KpnI restriction enzyme cutting site and the XhoI restriction enzyme cutting site of the vector pcDNA3.0 to obtain plasmid pcDNA3.0-PB1.

The double-stranded DNA molecule as shown in SEQ ID NO: 5 in the Sequence Listing was inserted between the KpnI restriction enzyme cutting site and the XhoI restriction enzyme cutting site of the vector pcDNA3.0 to obtain plasmid pcDNA3.0-PB2.

The double-stranded DNA molecule as shown in SEQ ID NO: 7 in the Sequence Listing was inserted between the KpnI restriction enzyme cutting site and the XhoI restriction enzyme cutting site of the vector pcDNA3.0 to obtain plasmid pcDNA3.0-NP.

II. Rescue of Viruses

1. 293T RIG-I KO cells were seeded in 60 mm dishes (1×10⁶ cells per dish), and cultured for 12 h.

2. Grouping and Treatment

Group 1: the plasmid pHH21-PA, the plasmid pHH21-PB1, the plasmid pHH21-PB2, the plasmid pHH21-HA, the plasmid pHH21-NP, the plasmid pHH21-NA, the plasmid pHH21-M, the plasmid pHH21-NS1R38A, the plasmid pcDNA3.0-PA, the plasmid pcDNA3.0-PB1, the plasmid pcDNA3.0-PB2 and the plasmid pcDNA3.0-NP (1 μg each) were mixed, and then co-transfected into the cells obtained in step 1 with the liposome 2000, and the cells were cultured at 37° C. for 6 h and then the culture system was replaced with the virus infection solution, and the cells were cultured at 37° C. for 72 h to harvest the supernatant and cells respectively.

Group 2: the only difference from Group 1 was that the plasmid pHH21-NS1R38A was replaced with the plasmid pHH21-NS1K41A.

Group 3: the only difference from Group 1 was that the plasmid pHH21-NS1R38A was replaced with the plasmid pHH21-NSR38A/K41A.

Group 4: the only difference from Group 1 was that the plasmid pHH21-NS1R38A was replaced with the plasmid pHH21-NS1.

Group 5: the only difference from Group 1 was that the plasmid pHH21-NS1R38A was not added.

The plasmid pHH21-PA, the plasmid pHH21-PB1, the plasmid pHH21-PB2, the plasmid pHH21-HA, the plasmid pHH21-NP, the plasmid pHH21-NA, the plasmid pHH21-M, the plasmid pHH21-NS1, the plasmid pcDNA3.0-PA, the plasmid pcDNA3.0-PB1, the plasmid pcDNA3.0-PB2 and the plasmid pcDNA3.0-NP were co-transfected into 293T RIG-I KO cells (i.e., Group 4 above), and the cells were cultured to obtain WSN virus A/WSN/1933 (H1N1) strain. The WSN virus A/WSN/1933 (H1N1) strain is also known as WSN virus wild type. Accordingly, after the plasmid pHH21-NS1 was replaced with the plasmid pHH21-NS1R38A, the resulting virus was a R38A single mutant virus.

Accordingly, after the plasmid pHH21-NS1 was replaced with the plasmid pHH21-NS1K41A, the resulting virus was a K41A single mutant virus. Accordingly, after the plasmid pHH21-NS1 was replaced with the plasmid pHH21-NSR38A/K41A, the resulting virus was a R38A/K41A double mutant virus.

The supernatant obtained in Group 1 was named R38A-F0 generation supernatant.

The supernatant obtained in Group 2 was named K41A-F0 generation supernatant.

The supernatant obtained in Group 3 was named R38A/K41A-F0 generation supernatant.

The supernatant obtained in Group 4 was named WT-F0 generation supernatant.

The supernatant obtained in Group 5 was named NC-F0 generation supernatant.

3. Detection of Supernatant Titer after One Blind Passage

\textcircled{1} (MDCK cells were seeded in 100-mm diameter dishes (7×10⁶ cells per dish), and cultured for 12 h.

\textcircled{2} After completing step \textcircled{1}, 500 μl of the supernatant harvested in step 2 was added to each dish, then cultured for 72 h, and the supernatant was harvested. The harvested supernatant was defined as F1 generation supernatant (containing the initially rescued F1 generation recombinant virus). The F1 generation supernatant was subjected to plaque identification. The base 10 logarithm of the number of plaques per ml of supernatant was the titer.

The F1 generation supernatant obtained from the R38A-F0 generation supernatant in step 3 was named R38A-F1 generation supernatant.

The F1 generation supernatant obtained from the K41A-F0 generation supernatant in step 3 was named K41A-F1 generation supernatant.

The F1 generation supernatant obtained from the R38A/K41A-F0 generation supernatant in step 3 was named R38A/K41A-F1 generation supernatant.

The F1 generation supernatant obtained from the WT-F0 generation supernatant in step 3 was named WT-F1 generation supernatant.

The F1 generation supernatant obtained from the NC-F0 generation supernatant in step 3 was named NC-F1 generation supernatant.

The titer of the R38A-F1 generation supernatant was 4.1±0.1. The titer of the K41A-F1 generation supernatant was 4.5±0.2. The titer of the R38A/K41A-F1 generation supernatant was 3.3±0.1. The titer of the WT-F1 generation supernatant was 5.6±0.1. The titer of the NC-F1 generation supernatant was 0, that is, it was unable to form plaques in MDCK cells.

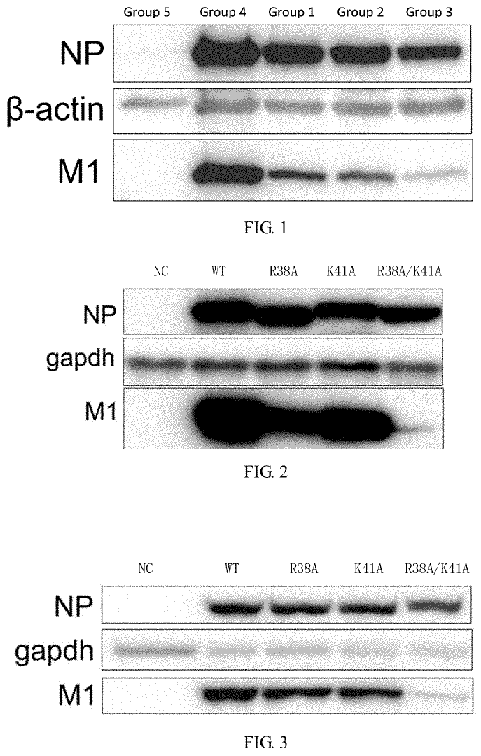

4. The cells obtained in step 2 were crushed and then subjected to western blot (to detect the expression of major viral proteins). In western blot, the primary antibody used to detect the NP protein was a polyclonal antibody against the NP protein; the primary antibody used to detect the M1 protein was a monoclonal antibody against the M1 protein. The western blot results are shown in FIG. 1. The two important proteins (NP protein and M1 protein) of influenza virus in the harvested cells of from Group 1 to Group 4 could be normally expressed.

### Example 2. Identification of Limited Replication Characteristics of Recombinant Virus (Blind Passaged on MDCK Cells)

The F1 generation supernatants prepared in Example 1 (the R38A-F1 generation supernatant, the K41A-F1 generation supernatant, the R38A/K41A-F1 generation supernatant, the WT-F1 generation supernatant, the NC-F1 generation supernatant) were subjected to the following steps, respectively:

1. Blind Passage

1. Two hundred microliters of F1 generation supernatant was added to 60 mm dishes (1×10⁶ MDCK cells were plated in each dish), and cultured for 72 h, and then the supernatant and cells were harvested separately. The harvested supernatant was defined as F2 generation supernatant. For ease of description, the harvested cells were named F2 cells.

2. Two hundred microliters of the F2 generation supernatant was added to 60 mm dishes (1×10⁶ MDCK cells were plated in each dish), and cultured for 72 h, and then the supernatant and cells were harvested separately. The harvested supernatant was defined as F3 generation supernatant. For ease of description, the harvested cells were named F3 cells.

3. Two hundred microliters of the F3 generation supernatant was added to 60 mm dishes (1×10⁶ MDCK cells were plated in each dish), and cultured for 72 h, and then the supernatant and cells were harvested separately. The harvested supernatant was defined as F4 generation supernatant. For ease of description, the harvested cells were named F4 cells.

The F2 generation supernatant, the F3 generation supernatant and the F4 generation supernatant obtained from the R38A-F1 generation supernatant by the above steps were successively named R38A-F2 generation supernatant, R38A-F3 generation supernatant and R38A-F4 generation supernatant.

The F2 generation supernatant, the F3 generation supernatant and the F4 generation supernatant obtained from the K41A-F1 generation supernatant by the above steps were successively named K41A-F2 generation supernatant, K41A-F3 generation supernatant and K41A-F4 generation supernatant.

The F2 generation supernatant, the F3 generation supernatant and the F4 generation supernatant obtained from the R38A/K41A-F1 generation supernatant by the above steps were successively named R38A/K41A-F2 generation supernatant, R38A/K41A-F3 generation supernatant and R38A/K41A-F4 generation supernatant.

The F2 generation supernatant, the F3 generation supernatant and the F4 generation supernatant obtained from the WT-F1 generation supernatant by the above steps were successively named WT-F2 generation supernatant, WT-F3 generation supernatant and WT-F4 generation supernatant.

The F2 generation supernatant, the F3 generation supernatant and the F4 generation supernatant obtained from the NC-F1 generation supernatant by the above steps were successively named NC-F2 generation supernatant, NC-F3 generation supernatant and NC-F4 generation supernatant.

II. The F2 generation supernatants, the F3 generation supernatants and the F4 generation supernatants were respectively subjected to plaque identification. The base 10 logarithm of the number of plaques per ml of supernatant was the titer.

The titers of the R38A-F2 generation supernatant, the R38A-F3 generation supernatant and the R38A-F4 generation supernatant were 4.0, 3.8 and 3.6, respectively.

The titers of the K41A-F2 generation supernatant, the K41A-F3 generation supernatant and the K41 A-F4 generation supernatant were 4.5, 4.3 and 4.3, respectively.

The titers of the R38A/K41A-F2 generation supernatant, the R38A/K41A-F3 generation supernatant and the R38A/K41A-F4 generation supernatant were 2.5, 2.0 and 0, respectively.

The titers of the WT-F2 generation supernatant, the WT-F3 generation supernatant and the WT-F4 generation supernatant were 6.0, 6.2 and 6.1, respectively.

The titers of the NC-F2 generation supernatant, the NC-F3 generation supernatant and the NC-F4 generation supernatant were 0, 0 and 0, respectively.

III. The F2 cells, the F3 cells, and the F4 cells were subjected to the following steps: crushed and then subjected to western blot (to detect the expression of major viral proteins). In western blot, the primary antibody used to detect the NP protein was a polyclonal antibody against the NP protein; the primary antibody used to detect the M1 protein was a monoclonal antibody against the M1 protein. The western blot results of the F2 cells are shown in FIG. 2. The western blot results of the F3 cells are shown in FIG. 3.

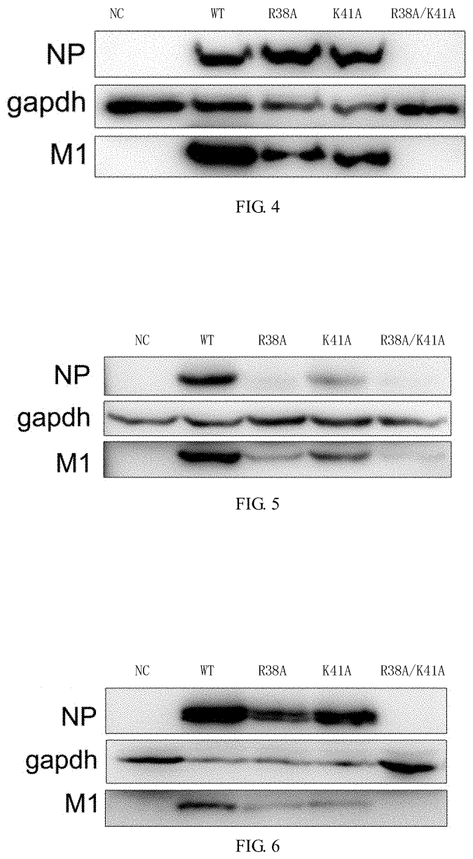

The western blot results of the F4 cells are shown in FIG. 4.

The above results indicated that the R38A/K41A double mutant virus lost its replication ability when blind passaged to the fourth generation.

### Example 3. Identification of Limited Replication Characteristics of Double Mutant Recombinant Virus (Passaged on A549 Cells)

The F1 generation supernatants prepared in Example 1 (the R38A-F1 generation supernatant, the K41A-F1 generation supernatant, the R38A/K41A-F1 generation supernatant, the WT-F1 generation supernatant, the NC-F1 generation supernatant) were subjected to the following steps, respectively:

1. Blind Passage

1. The Ft generation supernatant was added to 60 mm dishes (A549 cells were plated in dishes, MOI=0.001), and cultured for 72 h, and then the supernatant and cells were harvested separately. The harvested supernatant was defined as F2 generation supernatant. For ease of description, the harvested cells were named F2 cells.

2. Two hundred microliters of the F2 generation supernatant was added to 60 mm dishes (1×10⁶ A549 cells were plated in each dish), and cultured for 72 h, and then the supernatant and cells were harvested separately. The harvested supernatant was defined as F3 generation supernatant. For ease of description, the harvested cells were named F3 cells.

3. Two hundred microliters of the F3 generation supernatant was added to 60 mm dishes (1×10⁶ A549 cells were plated in each dish), and cultured for 72 h, and then the supernatant and cells were harvested separately. The harvested supernatant was defined as F4 generation supernatant. For ease of description, the harvested cells were named F4 cells.

The F2 generation supernatant, the F3 generation supernatant and the F4 generation supernatant obtained from the R38A-F1 generation supernatant by the above steps were successively named R38A-F2 generation supernatant, R38A-F3 generation supernatant and R38A-F4 generation supernatant.

The F2 generation supernatant, the F3 generation supernatant and the F4 generation supernatant obtained from the K41A-F1 generation supernatant by the above steps were successively named K41A-F2 generation supernatant, K41A-F3 generation supernatant and K41A-F4 generation supernatant.

The F2 generation supernatant, the F3 generation supernatant and the F4 generation supernatant obtained from the R38A/K41A-F1 generation supernatant by the above steps were successively named R38A/K41A-F2 generation supernatant, R38A/K41A-F3 generation supernatant and R38A/K41A-F4 generation supernatant.

The F2 generation supernatant, the F3 generation supernatant and the F4 generation supernatant obtained from the WT-F1 generation supernatant by the above steps were successively named WT-F2 generation supernatant, WT-F3 generation supernatant and WT-F4 generation supernatant.

The F2 generation supernatant, the F3 generation supernatant and the F4 generation supernatant obtained from the NC-F1 generation supernatant by the above steps were successively named NC-F2 generation supernatant, NC-F3 generation supernatant and NC-F4 generation supernatant.

II. The F2 generation supernatants, the F3 generation supernatants and the F4 generation supernatants were respectively subjected to plaque identification. The base 10 logarithm of the number of plaques per ml of supernatant was the titer.

The titers of the R38A-F2 generation supernatant, the R38A-F3 generation supernatant and the R38A-F4 generation supernatant were 3.7, 3.4 and 3.3, respectively.

The titers of the K41A-F2 generation supernatant, the K41A-F3 generation supernatant and the K41 A-F4 generation supernatant were 4.3, 4.2 and 4.0, respectively.

The titers of the R38A/K41A-F2 generation supernatant, the R38A/K41A-F3 generation supernatant and the R38A/K41A-F4 generation supernatant were 2.2, 0 and 0, respectively.

The titers of the WT-F2 generation supernatant, the WT-F3 generation supernatant and the WT-F4 generation supernatant were 5.8, 5.5 and 5.3, respectively.

The titers of the NC-F2 generation supernatant, the NC-F3 generation supernatant and the NC-F4 generation supernatant were 0, 0 and 0, respectively.

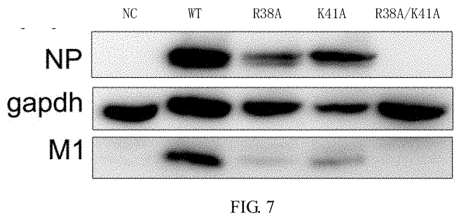

III. The F2 cells, the F3 cells, and the F4 cells were subjected to the following steps: crushed and then subjected to western blot (to detect the expression of major viral proteins). In western blot, the primary antibody used to detect the NP protein was a polyclonal antibody against the NP protein; the primary antibody used to detect the M1 protein was a monoclonal antibody against the M1 protein. The western blot results of the F2 cells are shown in FIG. 5. The western blot results of the F3 cells are shown in FIG. 6. The western blot results of the F4 cells are shown in FIG. 7.

The above results indicated that the R38A/K41A double mutant virus lost its replication ability when blind passaged to the third generation.

### Example 4. Double Mutant Recombinant Virus Infection in Mice

1. Weight and Mortality

After being anesthetized with ether, 5-week-old BALB/c mice weighing approximately 16 g were divided into 5 groups with 10 mice in each group, which were treated as follows:

Group 1: on day 0 of the test, 50 μl of the WT-F3 generation supernatant (with a virus content of 10³ PFU) prepared in Example 2 was inhaled by nasal inhalation, and then the body weight and mortality of the mice were observed daily.

Group 2: on day 0 of the test, 50 μl of the R38A/K41A-F3 generation supernatant (with a virus content of 10³ PFU) prepared in Example 2 was inhaled by nasal inhalation, and then the body weight and mortality of the mice were observed daily.

Group 3: on day 0 of the test, 50 μl of the WT-F2 generation supernatant (with a virus content of 10³ PFU) prepared in Example 3 was inhaled by nasal inhalation, and then the body weight and mortality of the mice were observed daily.

Group 4: on day 0 of the test, 50 μl of the R38A/K41A-F2 generation supernatant (with a virus content of 10³ PFU) prepared in Example 3 was inhaled by nasal inhalation, and then the body weight and mortality of the mice were observed daily.

Group 5: on day 0 of the test, 50 μl of sterilized PBS buffer was inhaled by nasal inhalation.

Relative body weight=body weight on a certain day+body weight on day 0 of the test×100%.

The results of the relative body weight and mortality are shown in Table 1.

TABLE 1

| | On day 3 of test | | On day 7 of test | | On day 14 of test | |
|---|---|---|---|---|---|---|
| | Body | | Body | | Body | |
| | weight | Mortality | weight | Mortality | weight | Mortality |
| Group 1 | 98.7% | 0 | 80.7% | 40% | 94.5% | 100% |
| Group 2 | 107.8% | 0 | 103.9% | 0 | 114.8% | 0% |
| Group 3 | 96.2% | 0 | 78.5% | 50% | 91.9% | 100% |
| Group 4 | 102.1% | 0 | 103.3% | 0 | 107.2% | 0% |
| Group 5 | 101.2% | 0 | 100.8% | 0 | 102.9% | 0% |

II. Virus-Serum Neutralizing Antibody Valence and Lung Virus Titer

After being anesthetized with ether, 5-week-old BALB/c mice weighing approximately 16 g were divided into 5 groups with 9 mice in each group, which were treated as follows:

Group 1: on day 0 of the test, 50 μl of the WT-F3 generation supernatant (with a virus content of 10³ PFU) prepared in Example 2 was inhaled by nasal inhalation.

Group 2: on day 0 of the test, 50 μl of the R38A/K41A-F3 generation supernatant (with a virus content of 10³ PFU) prepared in Example 2 was inhaled by nasal inhalation, and then the body weight and mortality of the mice were observed daily.

Group 3: on day 0 of the test, 50 μl of the WT-F2 generation supernatant (with a virus content of 10³ PFU) prepared in Example 3 was inhaled by nasal inhalation, and then the body weight and mortality of the mice were observed daily.

Group 4: on day 0 of the test, 50 μl of the R38A/K41A-F2 generation supernatant (with a virus content of 10³ PFU) prepared in Example 3 was inhaled by nasal inhalation, and then the body weight and mortality of the mice were observed daily.

Group 5: on day 0 of the test, 50 μl of sterilized PBS buffer was inhaled by nasal inhalation.

Three mice were sacrificed in each group on the 3rd, 7th and 14th days of the test, respectively. Serum was taken from the mice sacrificed on the 3rd, 7th and 14th days of the test to detect virus-serum neutralizing antibody valence (abbreviated as valence).

Lungs were taken from the mice sacrificed on the 3rd and 7th days of the test, and 1 g of tissue from each lung was homogenized in 1 ml ice-cold PBS buffer with a grinder. The homogenate was centrifuged at 5000 g for 10 min, and the supernatant was collected for plaque identification (the base 10 logarithm of the number of plaques per ml of supernatant was the titer.).

The results are shown in Table 2.

TABLE 2

| | | | | | On day 14 of |
|---|---|---|---|---|---|
| | On day 3 of test | | On day 7 of test | | test |
| | Valence | Titer | Valence | Titer | Valence |
| Group 1 | 1:16 | 6.34 ± 0.12 | 1:82 | 5.08 ± 0.19 | 1:224 |
| Group 2 | 1:19 | 4.85 ± 0.11 | 1:85 | 3.48 ± 0.12 | 1:203 |
| Group 3 | 1:18 | 5.85 ± 0.15 | 1:87 | 4.96 ± 0.11 | 1:202 |
| Group 4 | 1:17 | 4.69 ± 0.10 | 1:81 | 3.28 ± 0.06 | 1:198 |
| Group 5 | — | — | — | — | — |

III. Challenge Test

After being anesthetized with ether, 5-week-old BALB/c mice weighing approximately 16 g were divided into three groups with 6 mice in each group, which were treated as follows:

Group 1: on day 0 of the test, 50 μl of the R38A/K41A-F3 generation supernatant (with a virus content of 10³ PFU) prepared in Example 2 was inhaled by nasal inhalation; on day 17 of the test, 50 μl of the WT-F1 generation supernatant (with a virus content of 103.88 PFU, the volume was adjusted with DMEM medium) prepared in Example 1 was inhaled by nasal inhalation and the survival rate was calculated on day 32 of the test.

Group 2: on day 0 of the test, 50 μl of the R38A/K41A-F2 generation supernatant (with a virus content of 10³ PFU) prepared in Example 3 was inhaled by nasal inhalation; on day 17 of the test, 50 μl of the WT-F1 generation supernatant (with a virus content of 103.88 PFU, the volume was adjusted with DMEM medium) prepared in Example 1 was inhaled by nasal inhalation and the survival rate was calculated on day 32 of the test.

Group 3: on day 0 of the test, 50 μl of sterilized PBS buffer was inhaled by nasal inhalation; on day 17 of the test, 50 μl of the WT-F1 generation supernatant (with a virus content of 103.88 PFU, the volume was adjusted with DMEM medium) prepared in Example 1 was inhaled by nasal inhalation and the survival rate was calculated on day 32 of the test.

The survival rates of Group 1 and Group 2 were both 100%.

The survival rate of Group 3 was 0%.

### Example 5. Large-Scale Preparation

1. Two hundred microliters of the R38A/K41A-F1 generation supernatant prepared in Example 1 was added to 60 mm dishes (1×10⁶ Vero cells were plated in each dish), and cultured for 72 h, and then the supernatant was harvested. The harvested supernatant was defined as F2 generation supernatant.

2. Two hundred microliters of the F2 generation supernatant was added to 60 mm dishes (1×10⁶ Vero cells were plated in each dish), and cultured for 72 h, and then the supernatant was harvested. The harvested supernatant was defined as F3 generation supernatant.

3. Two hundred microliters of the F3 generation supernatant was added to 60 mm dishes (1×10⁶ Vero cells were plated in each dish), and cultured for 72 h, and then the supernatant was harvested. The harvested supernatant was defined as F4 generation supernatant.

4. Two hundred microliters of the F4 generation supernatant was added to 60 mm dishes (1×10⁶ Vero cells were plated in each dish), and cultured for 72 h, and then the supernatant was harvested. The harvested supernatant was defined as F5 generation supernatant.

5. Two hundred microliters of the F5 generation supernatant was added to 60 mm dishes (1×10⁶ Vero cells were plated in each dish), and cultured for 72 h, and then the supernatant was harvested. The harvested supernatant was defined as F6 generation supernatant.

6. Two hundred microliters of the F6 generation supernatant was added to 60 mm dishes (1×10⁶ Vero cells were plated in each dish), and cultured for 72 h, and then the supernatant was harvested. The harvested supernatant was defined as F7 generation supernatant.

7. Two hundred microliters of the F7 generation supernatant was added to 60 mm dishes (1×10⁶ Vero cells were plated in each dish), and cultured for 72 h, and then the supernatant was harvested. The harvested supernatant was defined as F8 generation supernatant.

8. Two hundred microliters of the F8 generation supernatant was added to 60 mm dishes (1×10⁶ Vero cells were plated in each dish), and cultured for 72 h, and then the supernatant was harvested. The harvested supernatant was defined as F9 generation supernatant.

9. Two hundred microliters of the F9 generation supernatant was added to 60 mm dishes (1×10⁶ Vero cells were plated in each dish), and cultured for 72 h, and then the supernatant was harvested. The harvested supernatant was defined as F10 generation supernatant.

The F2 generation supernatant, the F3 generation supernatant, the F4 generation supernatant, the F5 generation supernatant and the F10 generation supernatant were used as the supernatants to be tested, respectively, for plaque identification. The base 10 logarithm of the number of plaques per ml of supernatant was the titer.

The titer of the F2 generation supernatant was 3.34.

The titer of the F3 generation supernatant was 4.84.

The titer of the F4 generation supernatant was 4.34.

The titer of the F5 generation supernatant was 5.18.

The titer of the F10 generation supernatant was 4.51.

The results showed that Vero cells could be used for the passage and preparation of the virus solution.

### INDUSTRIAL APPLICATIONS

The influenza virus provided by the present invention can maintain replication ability for multiple generations by using Vero cells for passage, lose replication ability by using MDCK cells for passage to the fourth generation and lose replication ability by using A549 cells for passage to the third generation. Therefore, Vero cells can be used for passage, MDCK cells or A549 cells can be used to prepare vaccines (using the previous generation of viruses that have lost replication ability as vaccines), and mucosal immunization can be used. The present invention has great application value for the prevention and treatment of influenza virus.

What is claimed is:

1. A mutant protein, which is obtained by mutating two amino acid residues of NS1 protein of influenza virus as follows: the amino acid residue at position 38 is mutated from arginine to alanine, and the amino acid residue at position 41 is mutated from lysine to alanine.

2-13. (canceled)

14. The mutant protein of claim 1, wherein the influenza virus is influenza A virus.

15. A polynucleotide encoding the mutant protein of claim 1.

16. A recombinant plasmid, recombinant cells or recombinant virus containing the polynucleotide of claim 15.

17. The recombinant virus of claim 16, wherein the recombinant virus is obtained by mutating the codons encoding two amino acid residues of NS1 protein in the influenza virus genome as follows: the codon for the amino acid residue at position 38 is mutated from the arginine codon to an alanine codon, and the codon for the amino acid residue at position 41 is mutated from the lysine codon to an alanine codon.

18. The recombinant virus of claim 16, wherein the recombinant virus is prepared by the method comprising the following steps: ex vivo mammalian cells are co-transfected with plasmid pHH21-PA, plasmid pHH21-PB1, plasmid pHH21-PB2, plasmid pHH21-HA, plasmid pHH21-NP, plasmid pHH21-NA, plasmid pHH21-M, plasmid pHH21-NS1R38A/K41A, plasmid pcDNA3.0-PA, plasmid pcDNA3.0-PB1, plasmid pcDNA3.0-PB2 and plasmid pcDNA3.0-NP and then cultured to obtain the recombinant virus;
the plasmid pHH21-PA is a recombinant plasmid obtained by inserting the double-stranded DNA molecule as shown in SEQ ID NO: 3 in the Sequence Listing into the multiple cloning site of vector pHH21; the plasmid pHH21-PB1 is a recombinant plasmid obtained by inserting the double-stranded DNA molecule as shown in SEQ ID NO: 4 in the Sequence Listing into the multiple cloning site of vector pHH21; the plasmid pHH21-PB2 is a recombinant plasmid obtained by inserting the double-stranded DNA molecule as shown in SEQ ID NO: 5 in the Sequence Listing into the multiple cloning site of vector pHH21; the plasmid pHH21-HA is a recombinant plasmid obtained by inserting the double-stranded DNA molecule as shown in SEQ ID NO: 6 in the Sequence Listing into the multiple cloning site of vector pHH21; the plasmid pHH21-NP is a recombinant plasmid obtained by inserting the double-stranded DNA molecule as shown in SEQ ID NO: 7 in the Sequence Listing into the multiple cloning site of vector pHH21; the plasmid pHH21-NA is a recombinant plasmid obtained by inserting the double-stranded DNA molecule as shown in SEQ ID NO: 8 in the Sequence Listing into the multiple cloning site of vector pHH21; the plasmid pHH21-M is a recombinant plasmid obtained by inserting the double-stranded DNA molecule as shown in SEQ ID NO: 9 in the Sequence Listing into the multiple cloning site of vector pHH21; the plasmid pHH21-NSR38A/K41A is a recombinant plasmid obtained by inserting the polynucleotide of claim 3 into the multiple cloning site of vector pHH21; the plasmid pcDNA3.0-PA is a recombinant plasmid obtained by inserting the double-stranded DNA molecule as shown in SEQ ID NO: 3 in the Sequence Listing into the multiple cloning site of vector pcDNA3.0; the plasmid pcDNA3.0-PB1 is a recombinant plasmid obtained by inserting the double-stranded DNA molecule as shown in SEQ ID NO: 4 in the Sequence Listing into the multiple cloning site of vector pcDNA3.0; the plasmid pcDNA3.0-PB2 is a recombinant plasmid obtained by inserting the double-stranded DNA molecule as shown in SEQ ID NO: 5 in the Sequence Listing into the multiple cloning site of vector pcDNA3.0; the plasmid pcDNA3.0-NP is a recombinant plasmid obtained by inserting the double-stranded DNA molecule as shown in SEQ ID NO: 7 in the Sequence Listing into the multiple cloning site of vector pcDNA3.0.

19. The recombinant virus of claim 16, wherein the recombinant virus is influenza virus, or the recombinant virus is influenza A virus.

20. The recombinant virus of claim 16, wherein the recombinant virus has the properties of losing replication ability by using MDCK cells for passage to the fourth generation, and/or losing replication ability by using A549 cells for passage to the third generation, and/or maintaining replication ability for multiple generations by using Vero cells for passage.

21. A vaccine for influenza virus, the active ingredient of which is made of recombinant virus of claim 16.

22. The vaccine for influenza virus of claim 21, wherein the active ingredient of the vaccine for influenza virus is made of the previous generation of recombinant viruses that have lost replication ability.

23. The vaccine for influenza virus of claim 22, wherein the previous generation of recombinant viruses that have lost replication ability are obtained by using mammalian cells to perform passage cultivation to limited times.

24. The vaccine for influenza virus of claim 23, wherein the mammalian cells are MDCK cells and/or A549 cells;
and/or the limited times is 3 times or 4 times.

25. The vaccine for influenza virus of claim 21, the immune way is mucosal immunization.

26. The vaccine for influenza virus of claim 21, wherein the method for preparing the said vaccine for influenza virus comprising the following steps:
the recombinant virus is passaged N times; N is a natural number greater than or equal to 3;
the method of the first passage is as follows: mammalian cells are infected with the recombinant virus of claim 4, and then cultured to collect the supernatant, which is a virus solution containing F1 generation virus;
the method of the second passage is as follows: mammalian cells are infected with the virus solution obtained from the previous passage, and then cultured to collect the supernatant, which is a virus solution containing F2 generation virus;
a series of passages is performed by repeating the method of the second passage;
in the N−1th passage, the collected supernatant has virus replication ability;
in the Nth passage, the collected supernatant does not have virus replication ability;
N is a natural number greater than or equal to 3;
the supernatant collected from the N−1th passage is used as the active ingredient of the vaccine for influenza virus.

27. The vaccine for influenza virus of claim 21, wherein the method for preparing the said vaccine for influenza virus comprising the following steps:
(1) MDCK cells are infected with the recombinant virus of claim 16, and then cultured to collect the supernatant, which is a F1 generation virus solution;
(2) MDCK cells are infected with the F1 generation virus solution, and then cultured to collect the supernatant, which is a F2 generation virus solution;
(3) MDCK cells are infected with the F2 generation virus solution, and then cultured to collect the supernatant, which is a F3 generation virus solution;
(4) the F3 generation virus solution is used as the active ingredient of the vaccine for influenza virus.

28. The vaccine for influenza virus of claim 21, wherein the method for preparing the said vaccine for influenza virus comprising the following steps:
(1) MDCK cells are infected with the recombinant virus of claim 16, and then cultured to collect the supernatant, which is a F1 generation virus solution;
(2) A549 cells are infected with the F1 generation virus solution, and then cultured to collect the supernatant, which is a F2 generation virus solution;
(3) the F2 generation virus solution is used as the active ingredient of the vaccine for influenza virus.

29. A method for preparing a vaccine for influenza virus, comprising the following steps:
the recombinant virus is passaged N times; N is a natural number greater than or equal to 3;
the method of the first passage is as follows: mammalian cells are infected with the recombinant virus of claim 16, and then cultured to collect the supernatant, which is a virus solution containing F1 generation virus;
the method of the second passage is as follows: mammalian cells are infected with the virus solution obtained from the previous passage, and then cultured to collect the supernatant, which is a virus solution containing F2 generation virus;
a series of passages is performed by repeating the method of the second passage;
in the N−1th passage, the collected supernatant has virus replication ability;
in the Nth passage, the collected supernatant does not have virus replication ability;
N is a natural number greater than or equal to 3;
the supernatant collected from the N−1th passage is used as the active ingredient of the vaccine for influenza virus.

30. The method of claim 29, wherein the method comprise the following steps:
(1) MDCK cells are infected with the recombinant virus of claim 16, and then cultured to collect the supernatant, which is a F1 generation virus solution;
(2) MDCK cells are infected with the F1 generation virus solution, and then cultured to collect the supernatant, which is a F2 generation virus solution;
(3) MDCK cells are infected with the F2 generation virus solution, and then cultured to collect the supernatant, which is a F3 generation virus solution;
(4) the F3 generation virus solution is used as the active ingredient of the vaccine for influenza virus.

31. The method of claim 29, wherein the method comprise the following steps:
(1) MDCK cells are infected with the recombinant virus of claim 16, and then cultured to collect the supernatant, which is a F1 generation virus solution;
(2) A549 cells are infected with the F1 generation virus solution, and then cultured to collect the supernatant, which is a F2 generation virus solution;
(3) the F2 generation virus solution is used as the active ingredient of the vaccine for influenza virus.

32. A method for passaging the recombinant virus of claim 16, comprising the step of using VERO cells to perform passage cultivation.
